(19) United States
(12) Patent Application Publication
(10) Pub. No.: US 2018/0126374 A1
(43) Pub. Date: May 10, 2018

(54) STRIPS FOR QUANTITATIVE TRANSFER OF BIOCHEMICAL SAMPLES

(71) Applicant: ALPHA DIAGNOSTIC INTERNATIONAL, INC., San Antonio, TX (US)

(72) Inventor: Masarrat ALI, San Antonio, TX (US)

(21) Appl. No.: 15/572,577

(22) PCT Filed: May 6, 2016
(86) PCT No.: PCT/US16/31193

§ 371 Date: Nov. 8, 2017

Related U.S. Application Data
(60) Provisional application No. 62/158,898, filed on May 8, 2015.

(51) Int. Cl. B01L 3/00; G01N 1/40; G01N 33/52

(52) U.S. Cl. CPC B01L 3/5023; G01N 1/405; G01N 33/521; B01L 2300/0825; B01L 2200/141; B01L 2200/06; B01L 2300/069; B01L 2300/021

(57) ABSTRACT

Provided herein are devices and methods for facile, reliable, quantitative collection and transfer of liquid samples. In particular, the disclosure relates to strip-based systems for the quantitative collection and transfer of biological samples such as proteins, antibodies, DNA, bioanalytical reagents, and chemicals.

### CROSS-REFERENCE TO RELATED APPLICATIONS

This application claims the benefit of priority from U.S. Provisional Application No. 62/158,898, filed May 8, 2015, the disclosure of which is incorporated by reference herein in its entirety.

### FIELD OF THE INVENTION

Provided herein are devices and methods for the quantitative collection and transfer of biological samples. In particular, the disclosure relates to strip-based systems for the quantitative collection and transfer of biological samples such as proteins, antibodies, DNA, bioanalytical reagents, and chemicals.

### BACKGROUND

Collection, storage and shipping of biological or chemical samples are important process steps in many applications. Whether it relates to diagnostic testing, basic research, forensic evidence collection, disease research, or shipping samples to collaborators, the most widely used method of storing collected samples involves the use of some type of container such as a tube or vial. In most cases, the shipping of these samples involves the use of transporting in special boxes or containers. If the samples are perishable or temperature-sensitive, the samples are shipped refrigerated or frozen using insulated containers. The shipping costs can potentially be high when transporting perishable or temperature-sensitive samples.

A different problem arises when collecting samples in field settings or environments without adequate facilities or equipment for collection and storage of samples. For instance, in many remote locations or certain parts of the world, collection of blood samples from humans or animals for the specific purposes of biochemical and analytical screening can be difficult. Without access to equipment for accurate measurement of the quantity of sample being collected and facilities for proper storage of perishable samples and shipping for further analysis of the samples, the entire process can be challenging and expensive.

There are some practical solutions available for collection of liquid samples and drying them without the need for any special instrumentation. For instance, filter paper-based sample collection strips are available for blotting liquid samples and these are routinely used in neonatal units in hospitals and forensic testing laboratories. However, there is no mechanism to collect a defined quantity of liquid sample on these blotting papers without using a precision pipetting device. This can be a particular problem in sample collection efforts in field-based or remote settings. Moreover, there is no mechanism in these blotting paper systems that allows for quantitative elution of the dried material from the paper. Likewise, there is no mechanism to estimate the efficiency of the recovery process during the process of elution.

Yet another issue when dealing with large number of different samples from multiple species or types of samples or different geographical locations is the matter of proper labeling of samples to prevent any sample processing errors down the line. This can be very laborious and error prone if adequate care is not taken. Labeling processes can be performed efficiently with the use of automated label makers and barcode systems. But again, this requires access to special devices such as label-makers and barcode readers, all of which are difficult to source in, for example, remote areas or underserved parts of the world.

In summary, there is a need for a system that enables quantitative collection of liquid samples without the need for any specialized collection devices (such as a volumetric pipette or micropipette) and/or error-prone labeling processes.

### SUMMARY

The products and methods of the disclosure enable collection of liquid samples on a solid support matrix that enables drying of the samples, inactivation of any infectious or live biological material, easy shipping of the samples, and/or subsequent quantitative recovery of the dried material. The disclosure further provides a sample collection system that offers a simple, visually identifiable coding mechanism for tagging a sample to decrease sample collection and processing errors. The methods and devices disclosed herein are not limited to specific advantages or functionality.

In one aspect, the disclosure provides sample collection strips comprising: (a) a non-absorbent strip body; and (b) an absorbent pad attached to one end of the strip body; wherein the absorbent pad absorbs a predetermined amount of a sample fluid when exposed to the sample fluid. In some embodiments, the predetermined amount of sample fluid is accurate to within 10 microliters, to within 5 microliters, or to within 1 microliter. In some embodiments, the absorbent pad is made of or comprises one or more of PVDF, nitrocellulose, nylon, glass fiber, or a gel such as agarose or polyacrylamide. In another embodiment, the absorbent pad further comprises magnetic beads or nanoparticles. In some embodiments, the absorbent pad comprises a capture agent irreversibly (covalently) cross-linked to the matrix of the absorbent pad. In some embodiments, the capture reagent is an antibody, DNA, peptide, or receptor molecule.

In some embodiments, the absorbent pad comprises a predetermined amount of a non-reactive chemical marker, and wherein the non-reactive chemical marker elutes from the absorbent pad when exposed to an elution solvent. In some embodiments, the non-reactive chemical marker comprises a dye. In some embodiments, the predetermined amount of non-reactive chemical marker embedded in the absorbent pad is accurate to within 10%, to within 5%, or to within 1% of the predetermined amount.

In some embodiments, the absorbent pad of the sample collection strip comprises a sterilization agent capable of killing or inactivating infectious or living biological materials present in the sample. In some embodiments, the absorbent pad is sterile.

In some embodiments, the sample collection strips further comprise a combinatorial color code printed onto the strip base, wherein the combinatorial color code comprises one or more color tags, each color tag comprising one of a plurality of color options, and wherein the color options define a class of possible characteristics of the sample collection strip or the sample fluid, and each color option denotes a particular characteristic within the class. In some embodiments, the class of possible characteristics is the species from which the sample was collected, the type of liquid sample, the type of analyte of interest, the quantitative loading capacity of the strip, the amount of non-reactive chemical marker embedded in the absorbent pad, or the geographic origin of the sample.

In another aspect, the disclosure provides methods for the quantitative collection of a fluid sample comprising the steps of: (a) absorbing a predetermined amount of the fluid sample by immersing an absorbent pad of a sample strip of any of claims 1-8 into the fluid sample; and (b) drying the fluid sample absorbed by the sample strip. In some embodiments, the methods disclosed herein further comprise the step of (c) eluting the dried sample from the sample strip by contacting the strip with an elution solvent. In some embodiments, the methods disclosed herein further comprise the step of (d) analyzing the eluted sample.

These as well as other aspects, advantages, and alternatives, will become apparent to those of ordinary skill in the art by reading the following detailed description, with reference where appropriate to the accompanying drawings, and taken together with the accompanying claims.

### BRIEF DESCRIPTION OF THE DRAWINGS

The following detailed description of the embodiments of the present invention can be best understood when read in conjunction with the following drawings, in which:

FIG. 1 shows an exemplary rectangular strip according to the disclosure with the absorbent pad on one end.

FIG. 2A shows an exemplary method of dropping a fluid material onto the absorbent pad on the strip to saturate the pad, and FIG. 2B shows another exemplary method of inserting the top portion of the absorbent pad on the strip into a container of liquid to wick the liquid onto to the absorbent pad.

FIG. 3 shows an exemplary rectangular strip according to the disclosure with the absorbent pad on one end and with a chemical marker impregnated as a stripe on the distal end of the absorbent pad.

FIG. 4 shows an exemplary method of eluting a dried sample embedded in the strip along with the chemical marker from the absorbent pad into an elution solvent.

FIG. 5 shows an exemplary rectangular strip according to the disclosure with the absorbent pad on one end and with multiple combinatorial color tags on the end of the rectangular strip distal to the absorbent pad.

FIG. 6 is a flow chart illustrating the steps in the use of a sample collection strip according to the disclosure for absorbing a liquid and the subsequent elution of the dried material, according to some embodiments.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION

All publications, patents and patent applications cited herein are hereby expressly incorporated by reference for all purposes.

Provided herein are devices and methods useful for quantitative collection and/or transfer of liquid biological samples. In particular, the devices and methods disclosed herein comprise a simple system that enables quantitative collection of liquid samples without the need for any specialized devices. These devices and methods also enable drying of an accurately known, predetermined amount of a liquid sample, facilitating easy shipping or transfer of the samples and subsequent quantitative recovery of the dried material. In some embodiments, the devices and methods further enable inactivation of any infectious, contaminating, or otherwise undesired living biological material preset in the sample. In some embodiments, the methods and devices disclosed herein further comprise a simple, visually-identifiable coding mechanism for tagging the sample contained therein, which facilitates prevention of sample collection and processing errors.

Before describing the disclosed methods and devices in detail, a number of terms will be defined. As used herein, the singular forms “a”, “an”, and “the” include plural referents unless the context clearly dictates otherwise. For example, reference to a “sample” means one or more samples.

It is noted that terms like “preferably,” “commonly,” and “typically” are not utilized herein to limit the scope of the claimed aspects and embodiments or to imply that certain features are critical, essential, or even important to the structure or function of the claimed aspects and embodiments. Rather, these terms are merely intended to highlight alternative or additional features that can or cannot be utilized in a particular embodiment.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Exemplary embodiments are described herein. It should be understood that the word “exemplary” is used herein to mean “serving as an example, instance, or illustration.” Any embodiment described herein as “exemplary” is not necessarily to be construed as preferred or advantageous over other embodiments. Further, those skilled in the art will understand that changes and modifications may be made to these embodiments without departing from the true scope and spirit of the invention, which is defined by the claims.

### Absorbent Pads

In one aspect, the disclosure provides sample collection strips comprising: (a) a non-absorbent strip body, and (b) an absorbent pad attached to one end of the strip body, wherein the absorbent pad absorbs a predetermined amount of a sample fluid when exposed to the sample fluid.

In some embodiments, the non-absorbent strip body comprises an impervious material, such as plastic, glass, or ceramic. The strip body material should be impervious and non-absorbent with respect to any of the solvents or reagents or samples that the strip will be exposed to under ordinary use. Non-limiting examples of plastics include polyethylene terephthalate (PET), polyethylene (PE), high-density polyethylene (HDPE), low-density polyethylene (LDPE), polyvinyl chloride (PVC), polyvinylidene chloride (PVDC), polypropylene (PP), polystyrene (PS), high-impact polystyrene (HIPS), polyamides (PA) such as nylon, acrylonitrile butadiene styrene (ABS), polycarbonate (PC), polyurethane (PU), polytetrafluoroethylene (PTFE), and blends or combinations thereof.

The absorbent pad may be made of any material capable of absorbing, holding, or retaining a liquid sample in a reversible manner, such that the sample can be later eluted from the absorbent pad. For some applications, the absorbent pad comprises a hydrophilic material. For some applications, the absorbent pad comprises a lipophilic or hydrophobic material. In some embodiments, the absorbent pad is made from or comprises glass fiber, PVDF, nitrocellulose, nylon, or any other suitable polymer or biopolymer, such as agarose or polyacrylamide. For example, the absorbent pad may be made of specially formulated, completely synthetic material such as glass fiber pad to provide contaminant-free sampling for sensitive applications, such as DNA-based testing. For some applications, the absorbent pad is pre-treated to minimize even miniscule loss of samples that can occur due to adherence or adsorption of the analyte of interest (such as protein or DNA), thereby preventing later elution of the analyte from the pad.

In some embodiments, the absorbent pad additionally comprises magnetic beads or nanoparticles. In some embodiments, the magnetic beads, nanoparticles, or the matrix of the absorbent pad is irreversibly (i.e., covalently) linked to a capture reagent. Capture reagents include, but are not limited to, antibodies, DNA, peptides, receptor molecules, etc. In such embodiments, the capture reagent in the absorbent pad preferentially captures species in the absorbed sample fluid. For example, the sample fluid may contain an analyte of interest that is present only at very low, typically undetectable concentrations. An absorbent pad comprising a capture reagent directed to that particular analyte would, when exposed to the sample fluid, preferentially capture the analyte and thus concentrate it in the absorbent pad, yielding a detectable amount of the analyte in the pad.

In some embodiments, the absorbent pad, along with the transfer strip as a whole, and optionally any packaging, container, or vessel in which the transfer strip is contained, is sterilized. Sterile transfer strips can then be used without risk of contaminating or infecting the biological sample placed on the transfer strip. Sterilization of the absorbent pad, transfer strip, and/or container may be accomplished by any sterilization method known in the art, such as, for example, heat and/or pressure sterilization, such as by using an autoclave; irradiation, such as exposure to ultraviolet or gamma radiation; chemical sterilization, etc.

In some embodiments, the absorbent pad comprises a sterilization (or disinfection) agent, such as a chemical or biological sterilization agent, which is capable of deactivating or killing infectious, contaminating, or otherwise undesired living biological materials within the sample, such as bacteria, viruses, and spores. Such embodiments may be employed, for example, when transporting samples collected from infected individuals or samples of infectious bio-organisms. In such cases, the costs and/or complexity of transport or shipping may be lowered by sterilizing the samples, since shipping of active biological samples is typically costlier and more complicated than shipping of inactive samples. The chemical or biological sterilization agent could include organic or inorganic chemical substances, ionic or non-ionic detergents, proteases, nucleases, enzymes, lipids or any other specific or non-specific biological or chemical substance known to kill or inactivate bio-organisms. Examples of non-specific chemical sterilization agents include aldehydes (e.g., formaldehyde, glutaraldehyde), halogens (e.g., iodine, chlorine), alcohols (e.g., ethanol, propanol, isopropanol), phenols (e.g., carbolic acid), oxidants (e.g., peracetic acid, hydrogen peroxide, potassium permanganate), surfactants, etc. In some embodiments, the sterilization agent is specifically tailored to inactivate a specific infectious bio-organism within the sample without killing or inactivating other bio-organisms within the sample.

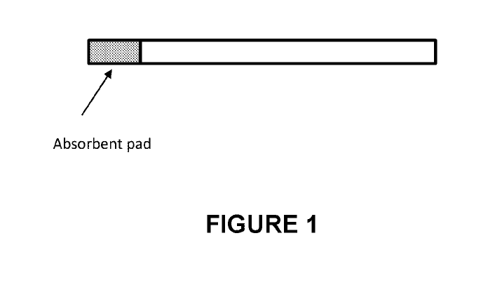

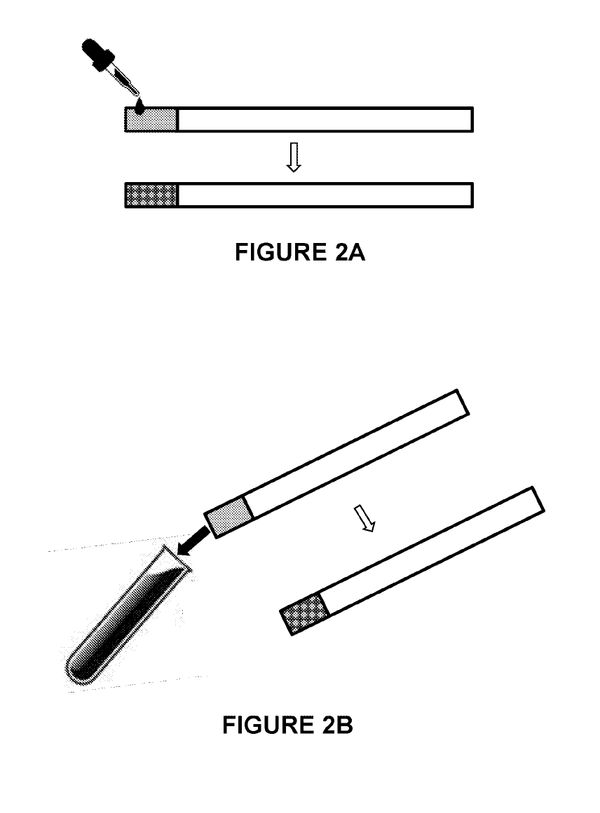

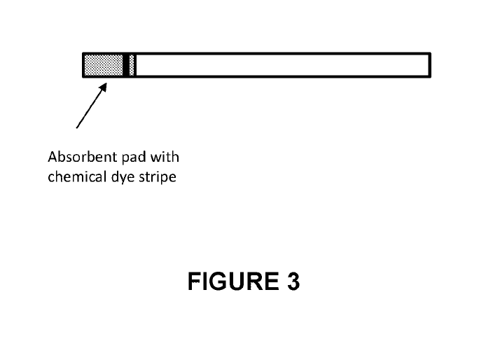

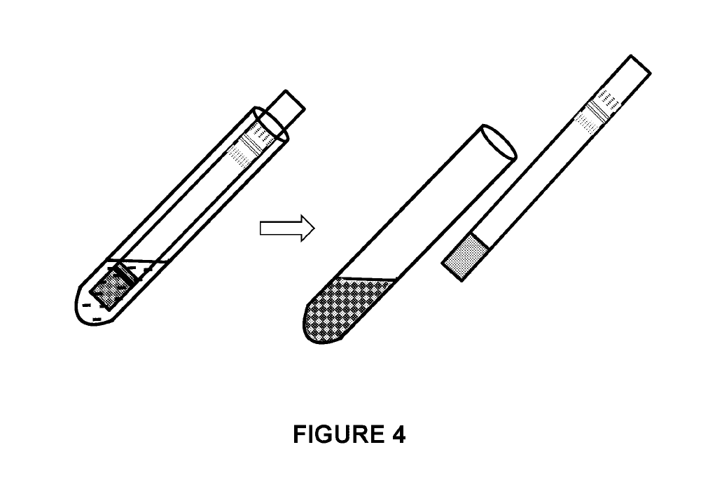

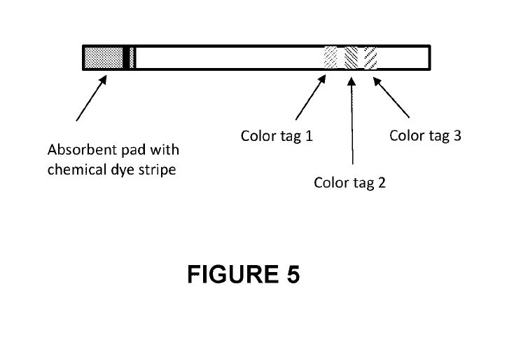

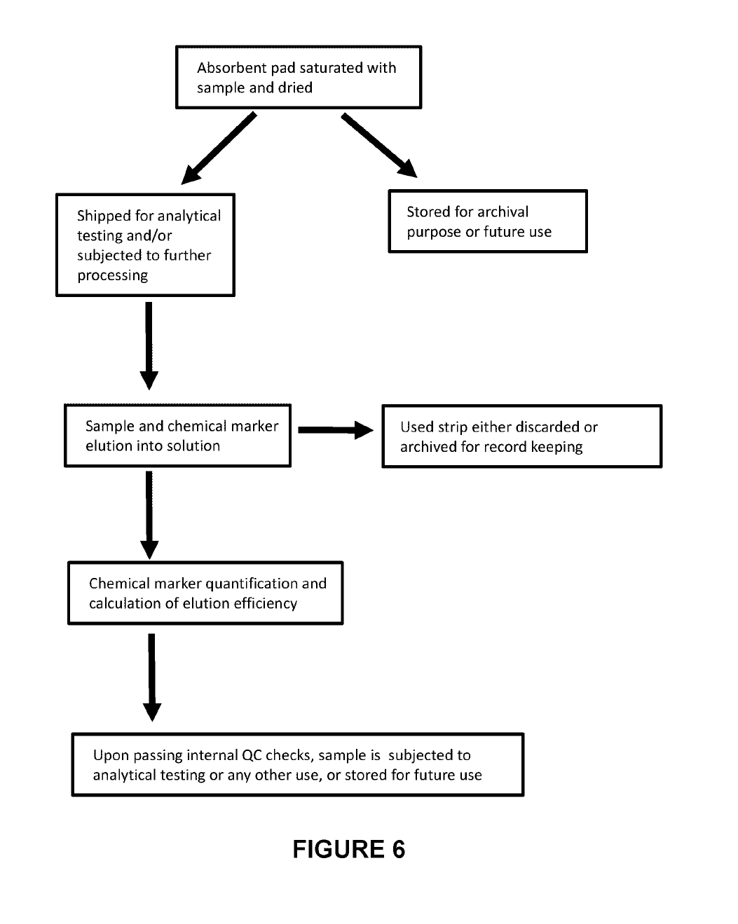

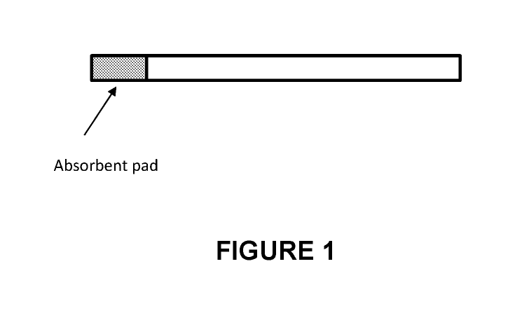

FIG. 1 illustrates an exemplary rectangular strip according to the disclosure with an absorbent pad at one end. This thin rectangular strip measuring approximately 0.5″ in width and 4″ in length is made of ordinary impervious material such as plastic. On one end of the strip, rectangular absorbent pads measuring 0.5″ wide and varying length ranging from 0.25″ to 1″ in length are adhered, fused, or otherwise attached to the strip. The purpose of having absorbent pads of varying lengths is to regulate the amount of liquid material that can be adsorbed by the pad. For instance, an absorbent pad measuring 0.5″ wide and 0.25″ long may only adsorb maximum amount of 10 microliters of a liquid sample, whereas a pad measuring 0.5″ wide and 1″ long may be able to adsorb maximum amount of 100 microliters of a liquid sample. The correlation between the maximum loading capacity and the precise dimensions of the absorbent pad are empirically determined using a wide range of different types of liquid samples.

By virtue of having absorbent pads of defined sizes that have a pre-determined maximum loading capacity of a range of liquid samples, this provides a simple system for loading specific amount of liquid material without the need for any measuring devices. As an example, a 10 microliter collection strip can be inserted into a vial of liquid to absorb 10 microliters of that liquid onto the strip. Once absorbed into the pad, the strip may be allowed to dry and is then ready for any further processing or transfer steps.

In some embodiments, the predetermined amount of sample fluid that the pad is capable of absorbing is accurate to within from about 0.1% to about 10% of the predetermined amount, or from about 0.5% to about 5% of the predetermined amount. In some embodiments, the predetermined amount is accurate to within about 5%, or about 4%, or about 3%, or about 2%, or about 1%, or about 0.5%, or about 0.1% of the predetermined amount. In some embodiments, the predetermined amount of sample fluid that the pad is capable of absorbing is accurate to within from about 0.1 microliters to about 50 microliters, or to within from about 0.1 microliters to about 10 microliters, or within from about 0.1 microliters to about 5 microliters. In some embodiments, the predetermined amount is accurate to within from about 1 microliter to about 5 microliters. In some embodiments, the predetermined amount is accurate to within 0.1 microliter, or to within 0.5 microliters, or to within 1 microliter, or to within 2 microliters, or to within 3 microliters, or to within 4 microliters, or to within 5 microliters.

As used herein with regard to the absorbent pad and fluid sample, the term “exposed to” refers to contacting the absorbent pad with an excess of fluid sample so that the absorbent pad is filled to capacity; i.e., such that the absorbent pad no longer absorbs additional liquid sample, even if exposed to more liquid sample. Since the liquid sample capacity of the absorbent pad is accurately pre-determined, exposing the absorbent pad to less liquid sample than can be absorbed by the pad results in an unknown amount of liquid sample in the pad. Only by contacting the absorbent pad with an excess of liquid sample will a predetermined amount of sample be absorbed by the pad.

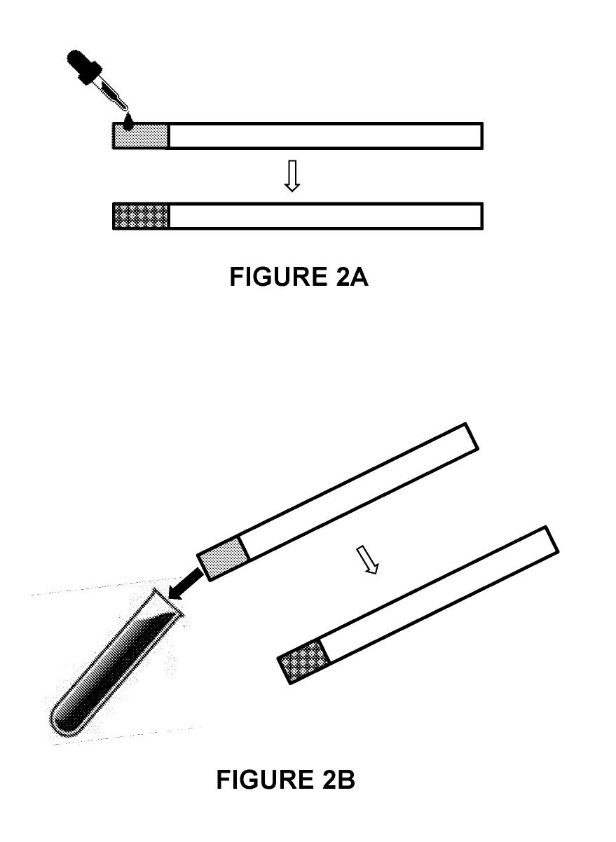

There are multiple methods of adsorbing a liquid onto the absorbent pad as shown in FIG. 2. FIG. 2A shows one method of adding a liquid substance using a dropper or an analytical pipette onto the pad. When the surface of the pad is saturated with the liquid, the addition of the liquid can be stopped. Because the pad is designed and optimized to adsorb only a predetermined amount of liquid, any excess liquid will flow out of the pad. Another exemplary method of adsorbing a liquid onto the pad is by allowing the liquid to wick onto the pad as shown in FIG. 2B. In this method, the proximal end of the pad of the collection strip is inserted into the container with the liquid. The liquid wicks onto the pad by capillary action and will rise all the way to top of the pad. At this point, the collection strip can be removed from the liquid. Another exemplary way of exposing the absorbent pad to a liquid sample (not depicted) is to fully immerse the absorbent pad into the liquid.

In some embodiments, after exposing the absorbent pad to the sample fluid, additional steps may be taken to remove excess fluid that has not been absorbed by the pad, but which remains on the surface of the pad due to surface tension or other phenomena. Examples of steps that may be taken to remove excess fluid include shaking or otherwise agitating excess fluid from the strip, blotting excess fluid, etc.

Regardless of the method used to adsorb the liquid on the transfer pad, in some embodiments, the next step involves drying the pad to allow the liquid to evaporate, leaving behind the analyte of interest within the matrix of the pad material. At this point, the collection strip is ready to be transported, archived, or to undergo additional processing steps as desired.

Drying of the strips may be performed at any temperature and pressure. In some embodiments, strips are dried at ambient temperature and atmospheric pressure. In some embodiments, strips are dried at elevated temperature. In some embodiments, the strips are dried at reduced (i.e. vacuum) pressure. In other embodiments, the strips are freeze-dried or lyophilized, i.e. at reduced temperature and reduced pressure. In all instances, the drying conditions will be determined based on the characteristics of the absorbed sample, such as the sample's stability at various temperatures and pressures.

### Chemical Markers

In some embodiments of the sample collection strips of the disclosure, the absorbent pad comprises a predetermined amount of a non-reactive chemical marker, wherein the non-reactive chemical marker elutes from the absorbent pad when exposed to an elution solvent.

As used herein, “non-reactive” refers to a chemical marker that does not bind to, react with, associate with, or otherwise interact with the analyte or analytes of interest in the fluid sample. In some embodiments, the chemical marker does not bind to, react with, associate with, or otherwise interact with any components of the fluid sample, such as any proteins, DNA, RNA, and/or other species present in the fluid sample. In general, the non-reactive chemical marker should not react with or otherwise interact with any component that disrupts the function of the marker or component. For example, if a plasma sample is collected then dried using a strip as disclosed herein so that a protein of interest within the plasma can be later analyzed, the non-reactive chemical embedded in the strip should not react or interact with the protein of interest; in some embodiments, the non-reactive chemical should also not react or interact with any other components in the plasma sample.

In some embodiments, the chemical marker comprises a dye, stain, or other analytical reagent capable of or suitable for detection via any known analytical method, such as spectrophotometry, mass spectroscopy, IR spectroscopy, UV/vis spectroscopy, fluorescence spectroscopy, Raman spectroscopy, liquid chromatography, gas chromatography, etc. Non-limiting examples of chemical markers include alizarin, alizarin red, alizarin yellow, amaranth, 1-amino-2-naphthol-4-sulfonic acid, aniline blue, azomethine, barium salts, brilliant ponceau, brilliant yellow, bromcresol green, bromcresol purple, bromphenol blue, bromthymol blue, calcein, calmagite, chloramine, chlorophenol red, cresol red, crystal violet, curcumin, 4,4′-dicarboxy-2,2′-biquinoline, 2′,7′-dichlorofluorescein, p-dimethylaminobenzalrhodanine, 4,4′-dimethyl-2,2′-bipyridine ferrous perchlorate, dimethylglyoxime, diphenylamine, s-diphenylcarbazone, 1,5-diphenylcarbohydrazide, 2,4-bis-(5,6-diphenyl-1,2,4-triazin-3-yl)pyridine, dipotassium pentacalcium dicalcein, dithizone, eosin, eriochrome black, eriochrome blue black, erioglaucine, eriogreen, fast sulphon black, ferric ammonium sulfate, fluorescein, furil, furildioxime, 2-hydroxy-1-(2-hydroxy-4-sulfo-1-naphthylazo-3-naphthoic acid), 7-hydroxy-4-methylcoumarin, hydroxy naphthol blue, metalphthalein, methyl calcein blue, methylene blue, methyl orange, methyl red, methyl purple, methylthymol blue, methyl violet, murexide, naphthol blue black, naphthol green, nathphylazoxine, nile blue, ninhydrin, phenol red, potassium chromate, resazurin, rhodamine, thorin, thymol blue, thymolphthalein, o-tolidine, variamine blue, xylenol orange, etc.

When the chemical marker is a dye, the identity of the chemical dye that is applied to the pad is known and can be used as tag to match up with a sample from certain organism or species. Each chemical dye has a unique spectral signature that can be detected using an analytical instrument or technique, for example, a spectrophotometer, a chromatograph, a mass spectrometer, etc. Other examples of chemical markers include radiolabeled compounds, or any compounds whose spectroscopic or other characteristics are known, and which can later be detected in the eluate by a known analytical method.

In some embodiments, the predetermined amount of non-reactive chemical marker embedded in the absorbent pad is accurately known. In some embodiments, the predetermined amount of non-reactive chemical marker embedded in the pad is accurate to within from about 0.1% to about 10% of the predetermined amount of chemical marker, or from about 0.5% to about 5% of the predetermined amount. In some embodiments, the predetermined amount is accurate to within about 5%, or about 4%, or about 3%, or about 2%, or about 1%, or about 0.5%, or about 0.1% of the predetermined amount. In some embodiments, the amount of chemical marker embedded in the absorbent pad is accurate to within about 0.001 mg to about 100 mg, or to within about 0.01 mg to about 10 mg, or to within about 0.01 mg to about 1 mg. In some embodiments, the amount of chemical marker embedded in the absorbent pad is accurate to within 0.01 mg or to within 0.1 mg.

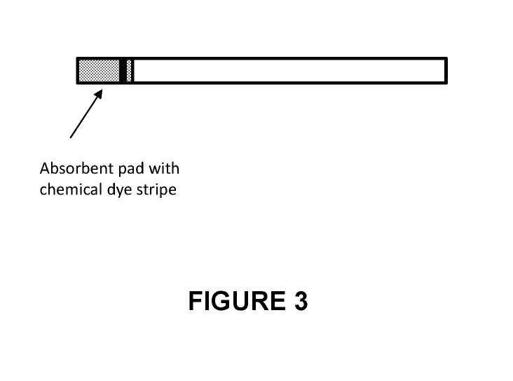

FIG. 3 illustrates an exemplary location of a chemical marker in the form of a chemical dye stripe impregnated into the absorbent pad. A measured amount a non-reactive chemical dye is applied to the absorbent paper pad. A different dye (in varying concentrations) is used in each strip that is intended for use with a sample from, for example, a specific species. For instance, a strip that will be used to adsorb a human serum sample may contain Dye A whereas a strip that will be used with bovine sample will contain Dye B. By virtue of using different dyes as species-specific sample tag, the chemical marker provides an internal control for confirming the species identity of the sample loaded onto the strip. For instance, if the strip containing Dye A tests positive for a bovine marker in the sample, clearly there may have been a sample mix-up error.

As used herein, the term “elution solvent” refers to any solvent capable of eluting the dried sample components in the dried absorbent pad into solution. In general, the elution solvent is selected based on the known solubility and/or stability of the analyte of interest and/or other components of the sample fluid. In some embodiments, where the analyte of interest and/or other dried sample components (and chemical marker) are water soluble, an aqueous or other hydrophilic or polar elution solvent may be used, such as, for example, water, buffer, saline, dimethyl sulfoxide (DMSO), ethanol, etc. In other embodiments, where the dried sample components and/or analyte of interest (and chemical marker) are not water soluble, a hydrophobic/lipophilic or non-polar elution solvent may be used, such as, for example, chloroform, dicholoromethane, cyclopentane, etc.

In some embodiments, the non-reactive chemical marker is used to determine the efficiency of the elution process of the dye and the dried sample into a liquid medium. Because the amount of chemical marker impregnated into the absorbent pad is accurately known, the degree of elution of the marker (along with the analyte of interest) can be determined by measuring the amount of eluted chemical marker in the elution solvent. By comparing the amount of chemical marker expected to be present in a given eluted sample against the actual amount of chemical marker present in the sample, this method offers a way to ascertain whether elution has occurred efficiently. In this manner, the dye functions as an internal control for ascertaining the fidelity and efficiency of the elution process and potentially other process related issues.

In some embodiments, the amount of elution solvent is accurately known, such that the total amount of eluted analyte or chemical marker can be calculated from the measured concentration of the analyte or marker.

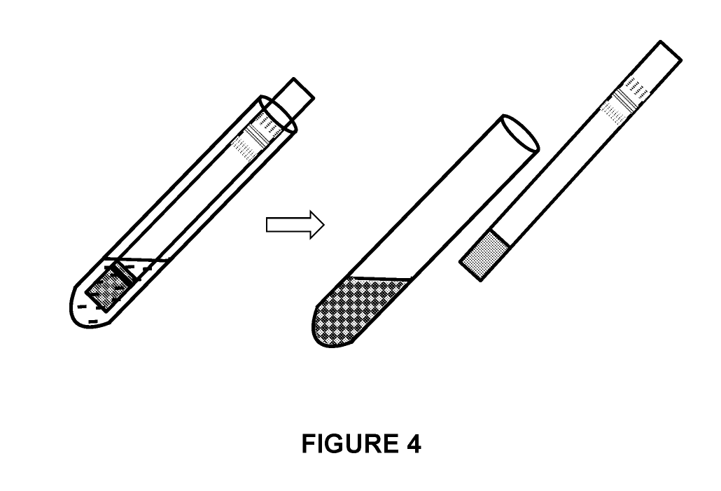

FIG. 4 shows an exemplary method of eluting the dried material (including the analyte(s) of interest) that is contained within the absorbent pad after sampling. As shown, the pad portion of the collection strip is inserted into a known quantity of elution solvent and the pad is allowed to incubate in the solution for a predetermined amount of time that has been optimized to allow nearly complete transfer of the dried material from the pad into the solution. It should be noted that along with the material that was applied to the pad, the chemical dye also transfers into the elution solution. At this point, the pad can be removed from the solution and either discarded or archived as desired. The efficiency of the elution process can be estimated by quantifying the amount of the chemical dye in the eluate. The detection of the chemical dye in the eluate is performed spectrophotometrically. As the concentration of the chemical dye that was originally applied to the pad on the collection strip is known, by estimating the concentration of the dye in the eluate, it is possible to quantify the efficiency of elution of the material from the pad.

### Combinatorial Color Codes

In some embodiments, the collection strips disclosed herein comprise “human-readable” combinatorial colored tags on one end of the strip along with the absorbent pad impregnated with “device-readable” dyes at the other end for the quantitative collection and/or transfer of a sample. The combinatorial colored tags enable ready identification of any functional information regarding the strip or sample collected using the strip.

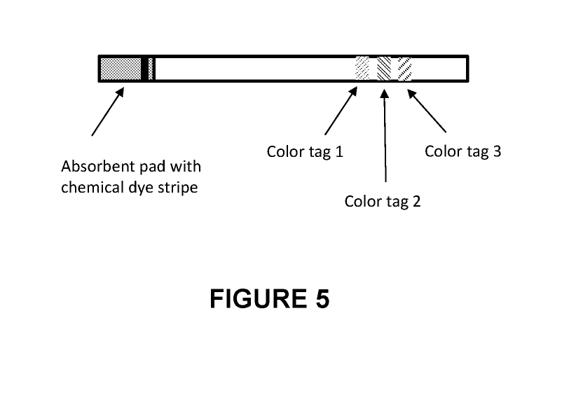

FIG. 5 illustrates the details and locations of the combinatorial colored tags on the collection strip. At the end of each strip opposite the absorbent pad is a series of color-coded visual tags (FIG. 5). Each strip may contain one or a plurality of color tags, for example up to 5-10 distinct color tags. The purpose of the tags is to visually identify and use the correct strip for the specific application. For instance, a strip may contain a color tag of 1 of several distinct color possibilities used to indicate the loading capacity of the absorbent pad on the other end of the strip. The same strip may contain in addition, a second color tag of 1 of possibly many colors that are entirely different from the first set of 4 colors. The functional purpose of the second color tag may be species-specific identity of the sample being absorbed into the pad. The same strip may also contain a third color tag of 1 of possibly many colors that are entirely different from the first and second set of colors. The functional purpose of the third color tag may be, for example, to encode the type of sample—whether it is DNA, protein, lipid, chemical or something else. The strip may also contain a fourth color tag of a set of colors distinct from the other three color tags, which encodes the location where the strip is to be used to collect a sample.

In this fashion, the combinatorial color tags provide an easy visual identification system for identification of the strip and the specific application for which the strip is being used for. In essence, it is much like a simple barcode system without the need for a barcode reader.

Thus, in some embodiments, the collection strips provided herein further comprise a combinatorial color code printed onto the strip base, wherein the combinatorial color code comprises one or more color tags, each color tag comprising one of a plurality of color options, and wherein the color options define a class of possible characteristics of the sample collection strip or of the sample fluid, and each color option denotes a particular characteristic within the class.

In some embodiments, the class of possible characteristics denoted by a given color tag is, for example, species from which the sample was collected, the type of liquid sample, the type of analyte of interest, the quantitative loading capacity of the strip, the type of chemical marker embedded in the absorbent pad, the amount of non-reactive chemical marker embedded in the absorbent pad, the geographic origin of the sample, etc.

Where the color tag denotes the species from which the sample was collected, different colors may be used to denote that the particular strip is to be used for, for example, human, bovine, porcine, murine, ovine, primate, or rat samples, or samples from any particular species.

Where the color tag denotes the type of liquid sample, different colors in that tag position may be used to denote that the particular strip is to be used for collecting, for example, serum samples, or blood samples, or urine samples, or plasma samples, or aqueous samples, or lipophilic samples, or tissue homogenate samples, or cell lysate samples, or samples in saline solution, or samples in buffer solution, or samples in organic solvent, etc.

Where the color tag denotes the type of analyte of interest, different colors in that tag position may be used to denote that the particular strip is to be used for, for example, protein samples, or DNA samples, or RNA samples, or enzyme samples, or membrane protein samples, or cytosolic protein samples, or lipid samples, or immunoglobulin samples, or IgA samples, or IgG samples, or IgE samples, IgM samples, or small molecule samples, etc.

Where the color tag denotes the quantitative sample loading capacity of the strip, different colors in that tag position may be used to denote that the absorbent pad of that particular strip will hold, when the absorbent pad has been saturated with liquid sample, a volume of, for example, 1 μL, or 2 μL, or 3 μL, or 4 μL, or 5 μL, or 10 μL, or 20 μL, or 25 μL, or 30 μL, or 40 μL, or 50 μL, or 60 μL, or 70 μL, or 75 μL, or 80 μL, or 90 μL, or 100 μL, or 150 μL, or 200 μL, or 250 μL, or 300 μL, or 400 μL, or 500 μL, or 1 mL, etc. In some embodiments, the strip is predetermined to hold a specific quantity of a particular type of sample denoted by a separate color tag.

Where the color tag denotes the type of non-reactive chemical marker embedded in the absorbent pad, different colors in that tag position may be used to denote that the chemical marker is, for example, a particular dye or analytical reagent, or is detectable by certain means, such as by UV/vis spectroscopy, or fluorescence spectroscopy, or infrared spectroscopy, or Raman spectroscopy, or mass spectroscopy, etc.

Where the color tag denotes the amount of non-reactive chemical marker impregnated into the absorbent pad, different colors in that tag position may be used to denote that the amount of chemical marker in the absorbent pad of that particular strip is, for example, about 0.01 mg, or about 0.02 mg, or about 0.03 mg, or about 0.04 mg, or about 0.05 mg, or about 0.1 mg, or about 0.2 mg, or about 0.3 mg, or about 0.4 mg, or about 0.5 mg, etc.

Where the color tag denotes the geographic origin of the sample, different colors in that tag position may be used to denote that the particular strip is to be used for, for example, samples originating in a particular country; or a particular geographic region, such as, for example, a state, a county, a province, a city, etc.; or a particular type of institution, such as, for example, a hospital, a university, a clinical research site, etc.; or a particular type of field location, such as, for example, a laboratory, a farm, a nature preserve, etc.; or a particular type of natural location, such as, for example, a stream, or a beach, or a river, or an ocean, or a mountain, or a pasture, or a desert, etc.

### Methods of Collecting Fluid Samples

In another aspect, the disclosure provides methods for quantitative collection of a fluid sample, comprising the steps of: (a) absorbing a predetermined amount of the fluid sample by exposing an absorbent pad of a sample collection strip as disclosed herein to the fluid sample; and (b) drying the fluid sample absorbed by the sample strip, wherein an analyte of interest in the fluid sample remains in the absorbent pad after drying.

In some embodiments, the methods disclosed herein further comprise the step of (c) eluting the dried sample from the sample strip by contacting the strip with an elution solvent. In some embodiments, the methods disclosed herein further comprise the step of (d) analyzing the eluted sample.

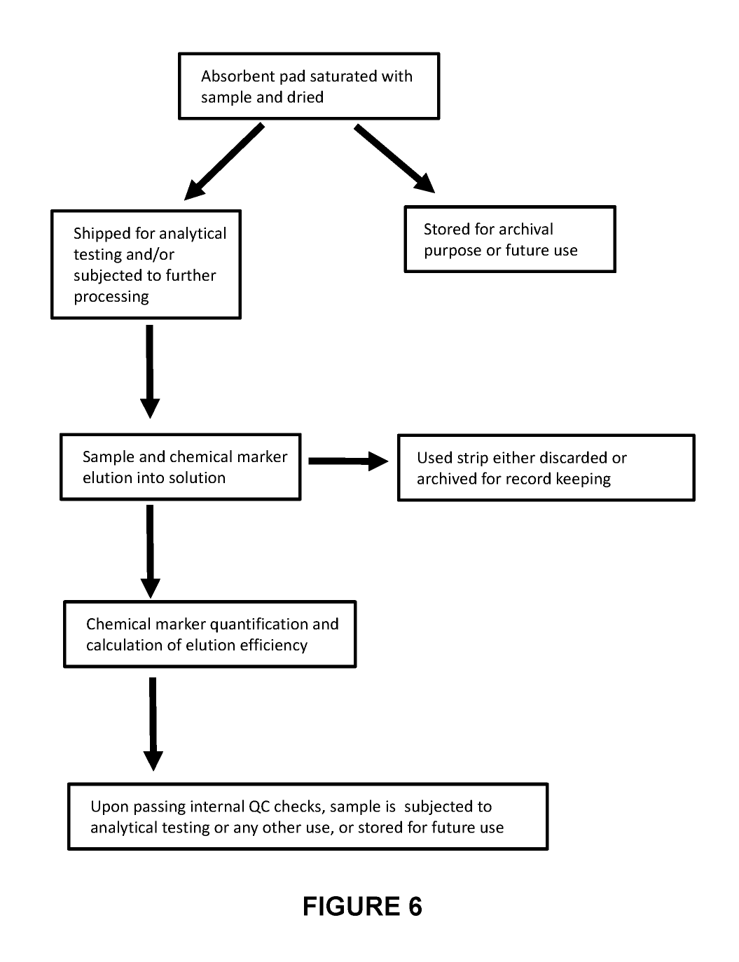

The entire process of using the collection strip in an exemplary embodiment is schematically illustrated in a flowchart shown in FIG. 6. The collection strip can be loaded with a sample using one of several methods—either adding the sample directly onto the pad or inserting the pad into a liquid sample that is in a container or tube. In an alternative method, the pad can also be bought into contact with a liquid sample that is not in a container. For instance, if the intent is to saturate the pad on the collection strip with blood from a finger prick, the pad would simply be bought into contact with the drop of blood. In any case, the pad saturated with the liquid sample is allowed to dry and then either stored for archival purposes, transferred to another location, and/or subjected to further processing and/or analysis.

If the next step is further bioanalytical processing, as shown in FIG. 6, the dried material on the pad is eluted out by contacting the absorbent pad with a suitable elution solvent. The efficiency of elution is then quantified by measuring the amount of non-reactive chemical marker present in the eluate and comparing it to the amount predicted to be in the eluate based on the predetermined amount of chemical marker initially impregnated into the absorbent pad. The next step is to perform the testing on the sample and either discard the collection strip or store it for further use.

It should be understood the arrangements and functions described herein are presented for purposes of example only, and that numerous variations are possible. For instance, elements can be added, omitted, combined, distributed, reordered, or otherwise modified.

1. A sample collection strip comprising:
(a) a non-absorbent strip body; and
(b) an absorbent pad attached to one end of the strip body;
wherein the absorbent pad absorbs a predetermined amount of a sample fluid when exposed to the sample fluid.

2. The sample collection strip of claim 1, wherein the predetermined amount of sample fluid is accurate to within 10%, to within 5%, or to within 1% of the predetermined amount.

3. The sample collection strip of claim 1, wherein the absorbent pad comprises PVDF, nitrocellulose, nylon, or glass fiber.

4. The sample collection strip of claim 1, wherein the absorbent pad comprises a predetermined amount of a non-reactive chemical marker, and wherein the non-reactive chemical marker elutes from the absorbent pad when exposed to an elution solvent.

5. The sample collection strip of claim 1, wherein the absorbent pad comprises magnetic beads or nanoparticles.

6. The sample collection strip of claim 5, wherein the absorbent pad further comprises a capture reagent covalently bonded to the absorbent pad, magnetic beads, or nanoparticles.

7. The sample collection strip of claim 6, wherein the capture reagent comprises an antibody, DNA, peptide, or receptor molecule.

8. The sample collection strip of claim 4, wherein the non-reactive chemical marker comprises a dye or a spectroscopically detectable analytical reagent.

9. The sample collection strip of claim 4, wherein the predetermined amount of non-reactive chemical marker embedded in the absorbent pad is accurate to within 1% of the predetermined amount of chemical marker.

10. The sample collection strip of claim 1, wherein the absorbent pad comprises a sterilization agent.

11. The sample collection strip of claim 1, which is sterile.

12. The sample collection strip of claim 1, further comprising a combinatorial color code printed onto the strip base,
wherein the combinatorial color code comprises one or more color tags, each color tag comprising one of a plurality of color options, and
wherein the color options define a class of possible characteristics of the sample collection strip or the sample fluid, and each color option denotes a particular characteristic within the class.

13. The sample collection strip of claim 12, wherein the class of possible characteristics is the species from which the sample was collected, the type of liquid sample, the type of analyte of interest, the quantitative loading capacity of the strip, the type of non-reactive chemical marker embedded in the absorbent pad, the amount of non-reactive chemical marker embedded in the absorbent pad, or the geographic origin of the sample.

14. A method for the quantitative collection of a fluid sample comprising the steps of:
(a) absorbing a predetermined amount of the fluid sample by immersing an absorbent pad of a sample strip of claim 1 into the fluid sample; and
(b) drying the fluid sample absorbed by the sample strip.

15. The method of claim 14, further comprising the step of (c) eluting the dried sample from the sample strip by contacting the strip with an elution solvent.
